(19) United States
(12) Patent Application Publication
(10) Pub. No.: US 2016/0017431 A1
(43) Pub. Date: Jan. 21, 2016

(54) Methods for Predicting EGFR Tyrosine Kinase Inhibitor Efficacy

(71) Applicant: Driver Group, San Francisco, CA (US)

(72) Inventors: Petros Giannikopoulos, San Francisco, CA (US); John St. John, Burlingame, CA (US); Joel Parker, Apex, NC (US); Trevor Bivona, San Francisco, CA (US)

(21) Appl. No.: 14/671,213

(22) Filed: Mar. 27, 2015

Related U.S. Application Data
(60) Provisional application No. 61/971,931, filed on Mar. 28, 2014.

(51) Int. Cl. C12Q 1/68; A61K 31/517; A61K 31/5377; G01N 33/574; G06F 19/24

(52) U.S. Cl. CPC C12Q 1/6886; G01N 33/57484; G06F 19/24; A61K 31/5377; A61K 31/517; C12Q 2600/158; C12Q 2600/106

(57) ABSTRACT

Provided herein are compositions and methods for predicting whether a subject with cancer will be responsive to treatment with an EGFR TKI and for the treatment of cancer in a subject.

### CROSS-REFERENCE TO RELATED APPLICATIONS

This application claims the benefit of U.S. Provisional Application No. 61/971,931 filed Mar. 28, 2014; the contents of which are hereby incorporated by reference.

### BACKGROUND

The identification and targeted therapeutic inhibition of certain molecular drivers of tumor growth has revolutionized the clinical management of many forms of cancer and has enabled personalized oncology. However, the clinical success of targeted cancer therapy has been limited due to heterogeneous patient responses and acquisition of drug resistance.

One example of targeted cancer therapy is the treatment of EGFR-mutant non-small cell lung cancer (NSCLC) using EGFR tyrosine kinase inhibitors (EFGR TKIs). EGFR-mutant NSCLC makes up approximately 10-35% of all instances of lung cancer (Pao and Girard, The Lancet Oncology 12:175-180 (2011)). The EGFR TKIs erlotinib and gefitinib are clinically approved first-line therapies in advanced stage EGFR-mutant NSCLC patients (Lynch et al., N. Engl. J. Med. 350:2129-2139 (2004); Paez, Science 304:1497-1500 (2004); Sordella et al., Science 305:1163-1167 (2004); Pao et al., Proc. Acad. Sci. U.S.A. 101:13306-13311 (2004)). Nonetheless, success with EGFR TKIs has been limited by acquisition of drug resistance in tumors, and virtually all patients progress within 9-14 months of initial treatment (Mok et al., N. Eng. J. Med. 361:948-957 (2009); Rosell et al., N. Eng. J. Med. 361:958-967 (2007)).

Several molecular events are known to contribute to EGFR TKI resistance by promoting tumor cell survival during EGFR TKI therapy, including the EGFR^{T790M} secondary resistance mutation and activation of several receptor and non-receptor kinases (Workman and Clarke, Cancer Cell 19:437-440 (2011); Zhang et al., Nature Genetics 44:852-860 (2012); Sequist et al., Science Translational Medicine 3:75ra26 (2011)). However, these known resistance-conferring alterations were identified using preclinical models and analysis of a small number of genes found in EGFR TKI resistant NSCLC clinical specimens. Thus, the current understanding of the molecular basis of EGFR TKI clinical resistance remains incomplete.

In light of the unpredictability of the responsiveness to EGFR TKI therapy in cancer patients, and especially in NSCLC patients, new compositions and methods for the prediction of EGFR TKI response and resistance, as well as for the treatment of EGFR mutant cancers are needed.

### SUMMARY

Provided herein are compositions and methods for predicting whether a subject with cancer will be responsive to treatment with an EGFR TKI and for the treatment of cancer in a subject.

In certain aspects, provided herein are methods for predicting whether a subject who has cancer will be responsive EGFR tyrosine kinase inhibitor (EGFR TKI) therapy. In some embodiments, the method includes analyzing a cancer cell sample from the subject to determine the expression level or gene copy number of NFKBIA. In some embodiments, the overexpression or elevated copy number of NFKBIA indicates that the subject will be responsive to EGFR TKI therapy.

In certain aspects, provided herein is a method of treating a cancer in a subject. In some embodiments the method includes administering an EGFR TKI to a subject who has a cancer in which NFKBIA is overexpressed and/or has an elevated copy number. In some embodiments, the method includes the steps of analyzing a cancer cell sample from the subject to determine the expression level or gene copy number of NFKBIA and administering an EGFR TKI to the subject if NFKBIA is overexpressed or has an elevated copy number in the sample. In some embodiments the EGFR TKI is gefitinib or erlotinib. In certain embodiments, the method includes the steps of analyzing a cancer cell sample from the subject to determine the expression level or gene copy number of NFKBIA and administering an EGFR TKI that is less prone to drug resistance to the subject if NFKBIA is not overexpressed or does not have an elevated copy number in the sample. In some embodiments, the EGFR TKI that is less prone to drug resistance is afatinib, dacomitinib or AP26113.

In some embodiments of the methods provided herein, the cancer carries an EGFR activating mutation (e.g., the cancer carries a mutation that results in the overexpression or constitutive activation of EGFR). In some embodiments, the cancer is a non-small cell lung cancer (NSCLC). In some embodiments, the cancer also carries a EGFR^{T790M} mutation. In some embodiments, the EGFR TKI is gefitinib, erlotinib, afatinib, lapatinib, dacomitinib, AP26113, cetuximab, panitumumab, zalutumumab, nimotuzumab necitumumab, RO5083945, ABT-806, or matuzumab. In some embodiments, the EGFR TKI is gefitinib, erlotinib or afatinib.

In some embodiments, any method can be used to determine the expression level or gene copy number of NFKBIA. In some embodiments, the analysis of the cancer cell sample comprises performing a nucleic acid amplification process. In some embodiments, the analysis of the sample comprises contacting the sample with a nucleic acid probe that hybridizes to a NFKBIA genomic DNA or mRNA sequence (e.g., a detectably labeled nucleic acid probe and/or a nucleic acid probe immobilized on a solid support). In some embodiments, the analysis of the subject sample comprises performing a deep sequencing assay. In some embodiments, the analysis of the subject sample comprises the step of contacting the sample with an anti-NFKBIA antibody or antigen binding fragment thereof. In some embodiments, the overexpression or elevated copy number of NFKBIA is relative to NFKBIA expression in non-cancer cells of the same tissue type as the cancer cells.

In certain aspects, provided herein is a method for determining whether a cancer in a subject is resistant to an EGFR TKI. In some embodiments, the method includes determining the expression level of a cell cycle/genome integrity gene set in a first cancer cell sample obtained before the subject began EGF TKI therapy. In some embodiments, the method includes determining the expression of the cell cycle/genome integrity gene set in a second cancer cell sample obtained after the subject had undergone EGF TKI therapy. In some embodiments, the method includes determining the expression of a stem cell/de-differentiation gene set in a first cancer cell sample. In some embodiments, the method includes determining the expression of the stem cell/de-differentiation gene set in a second cancer cell sample. In some embodiments, increased expression of the cell cycle/genome integrity gene set in the second cancer cell sample compared to the first cancer cell sample indicates that the cancer in the subject is resistant to the EGFR TKI. In some embodiments, decreased expression of the stem cell/de-differentiation gene set in the second cancer cell sample compared to the first cancer cell sample indicates that the cancer in the subject is resistant to the EGFR TKI. In some embodiments, increased expression of the cell cycle/genome integrity gene set in the second cancer cell sample compared to the first cancer cell sample and decreased expression of the stem cell/de-differentiation gene set in the second cancer cell sample compared to the first cancer cell sample indicates that the cancer in the subject is resistant to the EGFR TKI.

In certain aspects, provided herein is a method of treating a cancer in a subject. In some embodiments, the method includes obtaining a first cancer cell sample from the subject. In some embodiments, the method includes administering an EGFR tyrosine kinase inhibitor EGFR TKI to the subject. In some embodiments the EGFR TKI is gefitinib or erlotinib. In some embodiments, the method includes obtaining a second cancer cell sample from the subject. In some embodiments, the method includes determining the expression of a cell cycle/genome integrity and/or a stem cell/de-differentiation gene set in the first cancer cell sample and the second cancer cell sample. In some embodiments, the method includes the step of administering the EGFR TKI to the subject if the cell cycle/genome integrity gene set is not expressed at a higher level in the second cancer cell sample compared to the first cancer cell sample. In some embodiments, the method includes the step of halting the administration the EGFR TKI to the subject if the cell cycle/genome integrity gene set is expressed at a higher level in the second cancer cell sample compared to the first cancer cell sample. In certain embodiments, the method includes the step of administering an EGFR TKI that is less prone to resistance to the subject if the cell cycle/genome integrity gene set is expressed at a higher level in the second cancer cell sample compared to the first cancer cell sample. In some embodiments, the EGFR TKI that is less prone to drug resistance is afatinib, dacomitinib or AP26113. In some embodiments, the method includes the step of administering the EGFR TKI to the subject if the stem cell/de-differentiation gene set is expressed at a higher level in the second cancer cell sample compared to the first cancer cell sample. In some embodiments, the method includes the step of halting the administration the EGFR TKI to the subject if the stem cell/de-differentiation gene set is not expressed at a higher level in the second cancer cell sample compared to the first cancer cell sample. In some embodiments, the method includes the step of administering an EGFR TKI that is less prone to resistance to the subject if the stem cell/de-differentiation gene set is not expressed at a higher level in the second cancer cell sample compared to the first cancer cell sample. In some embodiments, the EGFR TKI that is less prone to drug resistance is afatinib, dacomitinib or AP26113. In some embodiments, the method includes the step of administering the EGFR TKI to the subject if the cell cycle/genome integrity gene set is not expressed at a higher level in the second cancer cell sample compared to the first cancer cell sample and the stem cell/de-differentiation gene set is expressed at a higher level in the second cancer cell sample compared to the first cancer cell sample. In some embodiments, the method includes the step of halting the administration the EGFR TKI to the subject if the cell cycle/genome integrity gene set is expressed at a higher level in the second cancer cell sample compared to the first cancer cell sample the stem cell/de-differentiation gene set is not expressed at a higher level in the second cancer cell sample compared to the first cancer cell sample. In some embodiments, the method includes the step of administering an EGFR TKI that is less prone to resistance to the subject if the cell cycle/genome integrity gene set is expressed at a higher level in the second cancer cell sample compared to the first cancer cell sample the stem cell/de-differentiation gene set is not expressed at a higher level in the second cancer cell sample compared to the first cancer cell sample. In some embodiments, the EGFR TKI that is less prone to drug resistance is afatinib, dacomitinib or AP26113.

In some embodiments, the cell cycle/genome integrity gene set comprise one or more genes selected from the group consisting of: WSB2, CKS18, PAICS, RRM1, HATQ, PCNA, CDK4, GINS2, SNRPB, HIST1H4C, PTMA, SLBP, PA2G4, MAD2L2, TMX1, MLF1P, MGAT2, STMN1, CDC6, C16orf61, TOPBP1, KIAA0090, ATP5G2, YY1, PRMT5, FEN1, DAXX, UNG, MCM3, CDK2, PRKDC, LMNB1, CCNB2, GINS1, WDR76, NUSAP1, CDT1, RBL1, CDK1, EZH2 and UMPS. In some embodiments the cell cycle/genome integrity gene set comprises WSB2, CKS18, PAICS, RRM1, HATQ, PCNA, CDK4, GINS2, SNRPB, HIST1H4C, PTMA, SLBP, PA2G4, MAD2L2, TMX1, MLF1P, MGAT2, STMN1, CDC6, C16orf61, TOPBP1, KIAA0090, ATP5G2, YY1, PRMT5, FEN1, DAXX, UNG, MCM3, CDK2, PRKDC, LMNB1, CCNB2, GINS1, WDR76, NUSAP1, CDT1, RBL1, CDK1, EZH2 and UMPS.

In some embodiments, the stem cell/de-differentiation gene set comprises at least one gene selected from the group consisting of SLK, CDKL5, PCDHB15, MARCH10, JPH2, PCDHB4, SOX5 and C19orf11. In some embodiments of the methods provided herein, the stem cell/de-differentiation gene set comprises SLK, CDKL5, PCDHB15, MARCH10, JPH2, PCDHB4, SOX5 and C19orf11.

In some embodiments, any method can be used to determine the expression of the gene sets. In some embodiments, expression of the gene sets is done using a process that includes performing a nucleic acid amplification process on the sample. In some embodiments, expression of the gene sets is done using a process that includes contacting the sample with a nucleic acid probes that hybridize to mRNA sequences encoded by the genes in the gene sets (e.g., a detectably labeled nucleic acid probe and/or a nucleic acid probe immobilized on a solid support). In some embodiments, expression of the gene sets is done using a process that includes performing a deep sequencing assay. In some embodiments, expression of the gene sets is done using a process that includes the step of contacting the sample with antibodies or antigen binding fragments that bind to proteins encoded by the genes of the gene set. In some embodiments, expression of the gene sets is determined using a gene expression or protein expression microarray. In some embodiments, expression of the integrity gene sets is determined by performing a deep sequencing assay.

In some embodiments, the cancer carries an EGFR activating mutation (e.g., the cancer carries a mutation that results in the overexpression or constitutive activation of EGFR). In some embodiments, the cancer is a non-small cell lung cancer (NSCLC). In some embodiments, the cancer also carries a EGFR^{T790M} mutation. In some embodiments, the EGFR TKI is gefitinib, erlotinib, afatinib, lapatinib, dacomitinib, AP26113, cetuximab, panitumumab, zalutumumab, nimotuzumab necitumumab, RO5083945, ABT-806, or matuzumab. In some embodiments, the EGFR TKI is gefitinib, erlotinib, afatinib, lapatinib, dacomitinib or AP26113. In some embodiments, the EGFR TKI is gefitinib or erlotinib. In some embodiments, the EGFR TKI is afatinib, dacomitinib or AP26113.

In some aspects, provided herein is a method for predicting whether a subject with a cancer carrying an EGFR activating mutation will develop an EGFR^{T790M} mutation. In some embodiments, the method includes determining the expression levels of SNORA53 and SDC2 in a cancer cell sample. In some embodiments, the method includes determining the expression level of a set of housekeeping genes in the cancer cell sample. In some embodiments, the method includes normalizing the expression level of SNORA53 and SDC2 against the mean expression of the set of housekeeping genes. In some embodiments, the method includes calculating a T790M score based on the normalized expression of SNORA53 and SDC2 to predict whether the subject will develop a EGFR^{T790M} mutation. In some embodiments, the T790M score is calculated based on the following equation:

$$T 790 M score = \frac{{e}^{- 349.07 - 13.50 \times SNORA 53 + 50.54 \times SDC 2}}{1 + {e}^{- 349.07 - 13.50 \times SNORA 53 + 50.54 \times SDC 2}}$$

wherein SNORA53 represents the normalized expression of SNORA53 and SDC2 represents the normalized expression of SDC2. In some embodiments, a T790M score of greater than 0.5 predicts that the subject will acquire a EGFR^{T790M} mutation. In some embodiments, the method includes administering an EGFR TKI to the subject if the subject is not predicted to develop a EGFR^{T790M} mutation.

In some aspects, provided herein is a method for treating a cancer. In some embodiments, the method comprises administering an EGFR TKI to a subject has a cancer with a T790M score of less than 0.5. In some embodiments, the method includes determining the expression levels of SNORA53 and SDC2 in a cancer cell sample. In some embodiments, the method includes determining the expression level of a set of housekeeping genes in the cancer cell sample. In some embodiments, the method includes normalizing the expression level of SNORA53 and SDC2 against the mean expression of the set of housekeeping genes. In some embodiments, the method includes calculating a T790M score based on the normalized expression of SNORA53 and SDC2 to predict whether the subject will develop a EGFR^{T790M} mutation. In some embodiments, the method includes administering to the subject an EGFR TKI if the subject is not predicted to develop a EGFR^{T790M} mutation. In some embodiments the EGFR TKI is gefinitib or erlotinib. In some embodiments, the method includes administering to the subject an EGFR TKI that is able to target EGFR carrying the T790M mutation if the subject is predicted to develop a EGFR^{T790M} mutation. In some embodiments, the EGFR TKI that is able to target EGFR carrying the T790M mutation is afatinib, dacomitinib or AP26113. In some embodiments, the T790M score is calculated based on the following equation:

$$T 790 M score = \frac{{e}^{- 349.07 - 13.50 \times SNORA 53 + 50.54 \times SDC 2}}{1 + {e}^{- 349.07 - 13.50 \times SNORA 53 + 50.54 \times SDC 2}}$$

wherein SNORA53 represents the normalized expression of SNORA53 and SDC2 represents the normalized expression of SDC2. In some embodiments, a T790M score of greater than 0.5 predicts that the subject will acquire a EGFR^{T790M} mutation.

In some embodiments of the methods provided herein, the set of housekeeping genes comprises one or more genes selected from the group consisting of C15orf24, C1orf43, CHMP2A, GPI, PSMB2, PSMB4, RAB7A, REEP5, SNRPD3, VCP and VPS29. In some embodiments the set of housekeeping genes comprises C15orf24, C1orf43, CHMP2A, GPI, PSMB2, PSMB4, RAB7A, REEP5, SNRPD3, VCP and VPS29.

In some embodiments, any method can be used to determine gene expression. In some embodiments, gene expression is determined by a process that comprises performing a nucleic acid amplification process on the sample. In some embodiments, gene expression is determined by a process that comprises contacting the sample nucleic acid probes that hybridize to a SNORA53 mRNA sequence (e.g., a nucleic acid probe, such as a detectably labeled nucleic acid probe and/or a nucleic acid probe immobilized on a solid support) and the expression level of SDC2 is determined by a process that comprises contacting the sample nucleic acid probes that hybridize to a SDC2 mRNA sequence (e.g., a nucleic acid probe, such as a detectably labeled nucleic acid probe and/or a nucleic acid probe immobilized on a solid support). In some embodiments, gene expression is determined by a process that comprises performing a deep sequencing assay. In some embodiments, gene expression is determined using a gene expression microarray or a protein expression microarray.

In some embodiments, the cancer carries an EGFR activating mutation (e.g., the cancer carries a mutation that results in the overexpression or constitutive activation of EGFR). In some embodiments, the cancer is a non-small cell lung cancer (NSCLC). In some embodiments, the cancer also carries a EGFR^{T790M} mutation. In some embodiments, the EGFR TKI is gefitinib, erlotinib, afatinib, lapatinib, dacomitinib, AP26113, cetuximab, panitumumab, zalutumumab, nimotuzumab necitumumab, RO5083945, ABT-806, or matuzumab. In some embodiments, the EGFR TKI is gefitinib, erlotinib, afatinib, lapatinib, dacomitinib or AP26113. In some embodiments, the EGFR TKI is gefitinib or erlotinib. In some embodiments, the EGFR TKI is afatinib, dacomitinib or AP26113.

In certain aspects, provided herein is a method of treating cancer in a subject, the method including administering to the subject an EGFR TKI. In some embodiments, the cancer has a phenotype that indicates that the cancer will be responsive to EGFR TKI therapy. In some embodiments, the cancer has a phenotype that indicates that it is unlikely to become resistant to EGFR TKI therapy. In some embodiments the cancer has a phenotype that indicates that it is unlikely to acquire an EGFR^{T790M} mutation. In some embodiments, the cancer overexpresses and/or has an elevated copy number of NFKBIA. In some embodiments, the expression of a cell cycle/genome integrity gene set did not increase in the cancer following administration of an EGFR TKI. In some embodiments, the expression of a stem cell/de-differentiation gene set increased in the cancer following administration of an EGFR TKI. In some embodiments, the cancer had a T790M score of under 0.5. In some embodiments, the EGFR TKI is gefitinib, erlotinib, afatinib, lapatinib, dacomitinib, AP26113, cetuximab, panitumumab, zalutumumab, nimotuzumab necitumumab, RO5083945, ABT-806, or matuzumab. In some embodiments, the EGFR TKI is gefitinib, erlotinib, afatinib, lapatinib, dacomitinib or AP26113. In some embodiments the EGFR TKI is gefitinib or erlotinib.

In certain aspects, provided herein is a method of treating cancer in a subject that includes administering to the subject an EGFR TKI that is less prone to resistance by the cancer. In some embodiments, the cancer has a phenotype that indicates that the cancer will be less responsive to EGFR TKI therapy. In some embodiments, the cancer has a phenotype that indicates that it is likely to become resistant to EGFR TKI therapy. In some embodiments the cancer has a phenotype that indicates that it is likely to acquire an EGFR^{T790M} mutation. In some embodiments, the cancer does not overexpress and/or does not have an elevated copy number of NFKBIA. In some embodiments, the expression of a cell cycle/genome integrity gene set increased in the cancer following administration of an EGFR TKI. In some embodiments, the expression of a stem cell/de-differentiation gene set did not increase in the cancer following administration of an EGFR TKI. In some embodiments, the cancer had a T790M score of over 0.5. In some embodiments, the EGFR TKI is that is less prone to resistance by the cancer is afatinib, PF299804 or AP26113.

### BRIEF DESCRIPTION OF THE DRAWINGS

FIG. 1 is a summary of somatic variants, copy number alterations and expression changes across the cohort for genes of interest and known mechanisms of acquired resistance. In acquired resistance biopsies, EGFR T790M seen in 6 cases, AXL and/or GAS6 upregulation in 5 patients, and MET amplification in 2 cases. A co-existing EML4-ALK fusion was observed in patient #8, with increased expression at resistance. A TPM3-ROS1 fusion was identified in the post-resistance biopsy of patient #11. * indicates normalized read counts for EML4-ALK. ** indicates EGFR copy number greater than in matched pre-treatment biopsy. *** ↑ indicates gene expression greater post-resistance; ↓ indicates gene expression lower post-resistance.

FIG. 2 is a gene set analysis of post-resistance versus pre-treatment gene expression profiles. (A) Comparison of gene expression profiles of post-resistance vs. pre-treatment cases reveals 3 significantly associated gene sets (q<0.2) corresponding to increased ERBB2 and FGFR signaling. Gray boxes denote cases with increased ERBB2 or FGFR1 mRNA expression in post-resistance biopsies. (B) Comparison of pre-treatment to post-resistance gene expression changes in cases that acquired EGFR^{T790M} at resistance (T790M+) versus those that did not (T790M−) revealed 60 significantly associated gene sets (q<0.2), 21 of which are shown in this heatmap and which represent genes associated with cell cycle, genome integrity maintenance, epigenetics, and stem cell/de-differentiation. T790M+ cases exhibited an increase in cell cycle, genome integrity and epigenetic gene expression, while T790M− cases showed an increase in stem cell signaling.

FIG. 3 shows that there is greater clonal divergence and genome copy number alteration in T790M+vs. T790M− cases. (A) Clonal divergence scores were derived for selected cases (cases that did not undergo whole genome amplification) by calculating the ratio of the sum of the number of mutations unique to pre-treatment and post-resistance to the number of shared mutations between pre and post

$$[ \frac{{MUT}_{unique \text{-} pre} -> {MUT}_{unique \text{-} post}}{{MUT}_{shared pre \text{-} post}} ] .$$

The number of unique and shared mutations for each case is depicted as cladograms where the length of each branch is proportional to the number of mutations. Collectively, the T790M+ cases demonstrate a trend towards higher degree of clonal divergence when compared to T790M− cases. (B) The percentage of each patient's pre-treatment and post-resistance tumor genome that is copy number altered is shown. When compared to T790M− patients, T790M+ patients demonstrate a higher degree of copy number alteration at resistance. (C) Comparison of pre-treatment to post-resistance mRNA expression changes in cases that acquired EGFR^{T790M} at resistance (T790M+) vs. those that did not (T790M−) reveals an increase in DNA repair pathway genes.

FIG. 4 shows progression-free survival according to T790M status. (A) T790M+ patients exhibit a trend towards better PFS. (B) Trend is accentuated when MET amplified patients are excluded from analysis, but statistical significance is not fully reached.

FIG. 5 shows a correlation of NFKBIA copy number and expression with EGFR TKI response. (A) Amplification of NFKBIA in post-resistance biopsies is positively correlated with TTP. (B) NFKBIA mRNA expression in post-resistance biopsies is positively correlated with TTP.

FIG. 6 shows progression-free survival correlation with NFKBIA copy number status and NFKBIA expression pre-treatment. (A) Amplification of NFKBIA in pre-TKI biopsies is positively correlated with PFS. (B) Level of mRNA expression of NFKBIA in pre-TKI biopsies is positively correlated with PFS.

### DETAILED DESCRIPTION

### General

Provided herein are compositions and methods for predicting whether a subject with cancer will be responsive to treatment with an EGFR TKI or will become resistance to EGFR TKI therapy. Also provided herein are compositions and methods for the treatment of cancer through administration of an EGFR-TKI to a subject who is likely to respond to treatment with that EGFR-TKI.

Despite some success in targeted cancer therapy using EGFR TKIs, lethal drug resistance occurs in most patients. The biological basis of the evolution of drug resistance is incompletely characterized. As disclosed herein, through integrated whole exome and transcriptome deep sequencing analysis of matched, paired tumor specimens obtained from EGFR-mutant NSCLC patients both before EGFR inhibitor therapy and at resistance novel diagnostic and prognostic markers of EGFR TKI efficacy and/or resistance were identified. For example, as described herein, the expression level and copy number of NFKBIA is predictive of patient responsiveness to EGFR TKI therapy, while certain gene expression profiles are predictive of EGFR TKI resistance. Additionally, expression of SNORA53 and SDC2, when normalized to a set of housekeeping genes, is predictive of the development of an EGFR^{T790M} mutation.

Thus, in certain aspects, provided herein are methods for predicting whether a subject with a cancer will be responsive EGFR tyrosine kinase inhibitor (EGFR TKI) therapy. In certain aspects, provided herein are methods for determining whether a cancer in a subject is or will become resistant to an EGFR TKI. In some aspects, provided herein are methods for predicting whether a subject with a cancer carrying an EGFR activating mutation will develop an EGFR^{T790M} mutation.

In certain aspects, provided herein are methods for treating cancer in a subject based on information provided using the diagnostic methods provided herein. For example, in some embodiments, an EGFR TKI is administered to a subject who was predicted to be responsive to the EGFR TKI and/or was predicted to be unlikely to acquire a resistance to the EGFR TKI. In some embodiments, an EGFR TKI is not administered to a subject who was predicted to not be responsive to the EGFR TKI and/or was predicted to acquire a resistance to the EGFR TKI. In some embodiments, an alternative therapeutic (e.g., a therapeutic for which resistance is unlikely) is administered to a subject if the subject was predicted to acquire resistance to an EGFR TKI. In some embodiments, an EGFR TKI that is less prone to resistance is administered to a subject who is predicted to acquire EGFR TKI resistance, such as an EGFR^{T790M} mutation.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, an antibody or antigen binding fragment thereof described herein.

The term “agent” is used herein to denote a chemical compound, a small molecule, a mixture of chemical compounds and/or a biological macromolecule (such as a nucleic acid, an antibody, an antibody fragment, a protein or a peptide). The activity of such agents may render them suitable as a “therapeutic agent” which is a biologically, physiologically, or pharmacologically active substance (or substances) that acts locally or systemically in a subject.

As used herein, the term “EGFR” refers to the epidermal growth factor receptor, the cell-surface receptor for the epidermal growth factor family of extracellular protein ligands. An exemplary human EGFR amino acid sequence is available as NCBI reference sequence NP_{—}005219.2, incorporated by reference herein. Mutations that lead to EGFR overexpression or overactivity can lead to cancer, including lung cancer such as non-small cell lung cancer. Other mutations in EGFR can lead to resistance to EGFR TKIs, including the EGFR^{T790M} mutation.

As used herein, the term “EGFR tyrosine kinase inhibitor” or “EGFR-TKI refers to any agent that inhibits the tyrosine kinase activity of EGFR. EGFR TKI can be, for example, small molecules, antibodies, antibody fragments, proteins, polypeptides. Examples of small molecule EGFR TKIs include gefitinib, erlotinib, afatinib, lapatinib, dacomitinib and AP26113. Examples of antibody EGFR TKI include cetuximab, panitumumab, zalutumumab, nimotuzumab necitumumab, RO5083945, ABT-806 and matuzumab

As used herein, the phrases “gene product” and “product of a gene” refers to a substance encoded by a gene and able to be produced, either directly or indirectly, through the transcription of the gene. The phrases “gene product” and “product of a gene” include RNA gene products (e.g. mRNA), DNA gene products (e.g. cDNA) and polypeptide gene products (e.g. proteins).

“Sample,” “subject sample,” or “biological sample” each refers to a collection of cells or cell components (e.g., proteins, DNA, RNA) obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject. In some embodiments, the sample is a “cancer cell sample.” As used herein, a cancer cell sample is a collection of cells that includes tumor and/or cancer cells or components of tumor and/or cancer cells, such as protein, DNA or RNA. The sample may also contain compounds that are not naturally intermixed with the tissue in nature such as preservatives, anticoagulants, buffers, fixatives, nutrients, antibiotics or the like.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Diagnostic and Prognostic Methods

Provided herein are diagnostic and/or prognostic methods for predicting the responsiveness of a cancer to an EGFR TKI therapy. In certain aspects, provided herein are methods for predicting whether a subject with a cancer will be responsive a EGFR tyrosine kinase inhibitor (EGFR TKI) therapy. In certain aspects, provided herein are methods for determining whether a cancer is or will become resistant to an EGFR TKI. In some aspects, provided herein are methods for predicting whether a cancer carrying will develop an EGFR^{T790M} mutation.

In some embodiments the methods provided herein can be applied to any cancer. In some embodiments, the cancer carries an EGFR activating mutation (e.g., the cancer carries a mutation that results in the overexpression or constitutive activation of EGFR). In some embodiments, the cancer is a non-small cell lung cancer (NSCLC), an anal cancer or glioblastoma multiforme. In some embodiments, the cancer is a cancer from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer carries a EGFR^{T790M} mutation.

In certain embodiments, the diagnostic and/or prognostic methods provided herein are used to inform patient care. For example, in some embodiments, the methods provided herein are used to determine whether a patient is a good candidate for EGFR TKI therapy. In some embodiments, a patient predicted to be responsive to an EGFR TKI is a good candidate for treatment with that EGFR TKI, whereas a patient who is predicted to have a high likelihood of acquiring resistance to an EGFR TKI is not a good candidate for treatment with that EGFR TKI. In some embodiments, a patient who is predicted to have a high likelihood of acquiring resistance to an EGFR TKI is a good candidate for treatment with a different EGFR TKI that is less prone to resistance.

In some embodiments the diagnostic and/or prognostic methods provided herein are used to inform the selection of the EGFR TKI to be administered to a subject. For example, in some embodiments a subject predicted to have a high likelihood of acquiring a EGFR^{T790M} mutation will be administered a therapeutic agent able to target EGFR^{T790M}, such as afatinib, dacomitinib or AP26113.

In certain embodiments, the method includes the analysis of a cancer cell sample from a subject. Such a sample can contain cancer cells from the subject or can contain components of the cancer cells or material derived from cancer cells. For example, a cancer cell sample can contain cancer cell components, such as cancer cell DNA, cancer cell RNA and/or cancer cell protein. A cancer cell sample can contain materials derived from a cancer cell, such as cDNA or amplification products generated by performing an amplification reaction on a cancer cell nucleic acid. In some embodiments, the method includes the step of obtaining the cancer cell sample from the subject. In some embodiments, the method includes steps for processing the cancer cell sample, such as by isolating cancer cell DNA, RNA and/or protein from other cancer cell components.

In certain embodiments, the processes described herein are computer-implemented. The processes may be implemented in software, hardware, firmware, or any combination thereof. The processes are preferably implemented in one or more computer programs executing on a programmable computer system including at least one processor, a storage medium readable by the processor (including, e.g., volatile and non-volatile memory and/or storage elements), and input and output devices. The computer system may comprise one or more physical machines or virtual machines running on one or more physical machines. In addition, the computer system may comprise a cluster of computers or numerous distributed computers that are connected by the Internet or other network.

Each computer program can be a set of instructions or program code in a code module resident in the random access memory of the computer system. Until required by the computer system, the set of instructions may be stored in another computer memory (e.g., in a hard disk drive, or in a removable memory such as an optical disk, external hard drive, memory card, or flash drive) or stored on another computer system and downloaded via the Internet or other network. Each computer program can be implemented in a variety of computer programming languages.

### NFKBIA as an Indicator of EGFR TKI Response

In certain aspects, provided herein are methods for predicting whether a subject with a cancer will be responsive EGFR TKI therapy based on expression level and/or copy number of NFKBIA. In some embodiments, the overexpression or elevated copy number of NFKBIA indicates that the subject will be responsive to EGFR TKI therapy.

NFKBIA (Nuclear Factor of Kappa Light Polypeptide Gene Enhancer in B Cells Inhibitor, Alpha) encodes a member of the NF-κB inhibitor family. The encoded protein interacts with REL dimers to inhibit NF-κB/REL complexes which are involved in the inflammatory response. As described herein, cancer subjects with increased expression and/or gene copy number of NFKBIA have an improved response to EGFR TKI therapy. The genomic DNA sequence of the NFKBIA gene is available as NCBI reference NC_{—}000014.8, which is hereby incorporated by reference. The mRNA sequence expressed by the NFKBIA gene is available as NCBI reference NM_{—}020529.2, which is hereby incorporated by reference. The amino acid sequence of the protein encoded by the NFKBIA gene is available as NCBI reference NP_{—}065390.1, which is hereby incorporated by reference.

In some embodiments, the method includes analyzing a cancer cell sample from the subject to determine the expression level or gene copy number of NFKBIA As used herein, the “expression level” of NFKBIA is the amount of NFKBIA expression product (e.g., mRNA or protein) present in the cancer cell sample on a per cell basis. As used herein, the “gene copy number” of NFKBIA is the number of genomic copies of the NFKBIA gene present in the cancer cell sample on a per cell basis.

In some embodiments of the methods provided herein, any method can be used to determine the expression level or gene copy number of NFKBIA. For example, gene copy number can be determined by northern blot, a nucleic acid amplification assay (e.g., PCR), a real-time nucleic acid amplification assay (e.g., real-time PCR), a deep sequencing assay, a dot blot, a nucleic acid microarray that recognizes genomic DNA or fluorescent microscopy (e.g., a fluorescent in situ hybridization assay). Gene expression level can be determined by detecting the amount of a gene expression product in the sample. For example, NFKBIA mRNA can be detected by northern blot, a nucleic acid amplification assay (e.g., RT-PCR), a real-time nucleic acid amplification assay (e.g., real-time RT-PCR), a deep sequencing assay, a dot blot or a gene expression microarray. Gene expression can also be determined by detecting the amount of NFKBIA protein in a sample by, for example, western blot, ELISA, FACS, fluorescent microscopy or a protein expression microarray.

In some embodiments, the analysis of the subject sample comprises performing a nucleic acid amplification process on the sample. In certain embodiments, the NFKBIA gene or a NKFKBIA expression product (e.g., mRNA) is amplified in the cancer cell sample using a nucleic acid amplification process. Examples of nucleic acid amplification processes include, but are not limited to, polymerase chain reaction (PCR), LATE-PCR a non-symmetric PCR method of amplification, ligase chain reaction (LCR), strand displacement amplification (SDA), transcription mediated amplification (TMA), self-sustained sequence replication (3SR), Qβ replicase based amplification, nucleic acid sequence-based amplification (NASBA), repair chain reaction (RCR), boomerang DNA amplification (BDA) and/or rolling circle amplification (RCA).

In some embodiments the analysis of the subject sample comprises contacting the sample with a nucleic acid probe that hybridizes to a NFKBIA genomic DNA, mRNA or cDNA sequence or an amplification product generated through the amplification of a NFKBIA genomic DNA, mRNA or cDNA sequence. In certain embodiments, the nucleic acid probe comprises a nucleic acid sequence that is complementary to a nucleic acid sequence of the NFKBIA genomic DNA, mRNA or cDNA sequence or an amplification product thereof. In some embodiments, the nucleic acid probe comprises at least 10, 15, 20, 25 or 30 nucleic acids that are complementary to a nu of nucleic acid sequence of the NFKBIA genomic DNA, mRNA or cDNA sequence or an amplification product thereof. In some embodiments, the nucleic acid probe comprises a detectable label. In some embodiments, the detectable label is a fluorescent label, a luminescent label, an enzymatic label or a radioactive label. In some embodiments the nucleic acid probe is a molecular beacon, a molecular torch, a TaqMan probe or a scorpion probe. In some embodiments, the nucleic acid probe is immobilized on a solid support. In certain embodiments, the solid support is a bead. In some embodiments, the solid support is a part of a nucleic acid microarray.

In some embodiments, the analysis of the subject sample comprises performing a deep sequencing assay. In a deep sequencing assay, the coverage of the sequencing assay is sufficient that the same nucleic acid region is sequenced multiple times. Deep sequencing therefore allows for the quantitation of the frequency at which a particular nucleic acid sequence appears in a sample. Nucleic acid sequencing processes that can be applied to deep sequencing include, but are not limited to, chain termination sequencing, sequencing by ligation, sequencing by synthesis, pyrosequencing, ion semiconductor sequencing, single-molecule real-time sequencing, 454 sequencing, and/or Dilute-‘N’-Go sequencing.

In some embodiments, the expression level of NFKBIA is determined by detecting the amount of NFKBIA protein in the sample. In some embodiments, the analysis of the subject sample comprises the step of contacting the sample with an anti-NFKBIA antibody or antigen binding fragment thereof. In some embodiments, the anti-NFKBIA antibody or antigen binding fragment thereof is linked (either directly or indirectly) to a detectable label. In some embodiments, the detectable label is a fluorescent label, a luminescent label, an enzymatic label or a radioactive label. In some embodiments, the anti-NFKBIA antibody or antigen binding fragment thereof is immobilized on a solid support. In certain embodiments, the solid support is a bead. In some embodiments, the solid support is a part of a protein microarray.

In some embodiments, the NFKBIA gene copy number is considered to be elevated if the gene copy number is greater than 2 copies per cell. In some embodiments the gene copy number is considered elevated if the copy number is greater than 2.1, 2.2, 2.3, 2.4, 2.5, 2.6, 2.7. 2.8, 2.9. 3, 3.5 or 4 copies per cell.

In some embodiments, the NFKBIA expression level is considered to be overexpressed if the NFKBIA expression level is higher than the NFKBIA expression level in a control cell population. In some embodiments, the control cell population is a population of non-cancer cells from the same tissue as the cancer cells. In some embodiments, the NFKBIA is overexpressed if the expression level is higher than the NFKBIA expression level in a cancer cell population of the same type that responds poorly to EGFR TKI therapy. In some embodiments, the NFKBIA is overexpressed if the expression level is comparable to or higher than the NFKBIA expression level in a cancer cell population of the same type that is responsive to EGFR TKI therapy.

### Gene Signatures of EGFR-TKI Resistance

In certain aspects, provided herein is a method for determining whether a cancer in a subject is resistant to an EGFR TKI based on changes in the expression level of a cell cycle/genome integrity gene set and/or a stem cell/de-differentiation gene set during the course of EGFR TKI therapy. In some embodiments, increases in the expression of the cell cycle/genome integrity gene set during the course of therapy indicates that the cancer in the subject is resistant to the EGFR TKI. In some embodiments, decreases expression of the stem cell/de-differentiation gene set during the course of therapy indicates that the cancer in the subject is resistant to the EGFR TKI. In some embodiments, increases in the expression of the cell cycle/genome integrity gene set and decreases in the expression of the stem cell/de-differentiation gene set during the course of therapy indicates that the cancer in the subject is resistant to the EGFR TKI. Increases or decreases in the expression of genes in a gene set refer to changes in the expression of the genes in aggregate. The individual expression levels of every gene in the gene set does not need to increase in order for the expression level of the gene set to increase in aggregate. Similarly, the individual expression levels of every gene in the gene set does not need to decrease in order for the expression level of the gene set to decrease in aggregate.

In some embodiments, the cell cycle/genome integrity gene set comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more genes selected from the group consisting of: WSB2, CKS18, PAICS, RRM1, HATQ, PCNA, CDK4, GINS2, SNRPB, HIST1H4C, PTMA, SLBP, PA2G4, MAD2L2, TMX1, MLF1P, MGAT2, STMN1, CDC6, C16orf61, TOPBP1, KIAA0090, ATP5G2, YY1, PRMT5, FEN1, DAXX, UNG, MCM3, CDK2, PRKDC, LMNB1, CCNB2, GINS1, WDR76, NUSAP1, CDT1, RBL1, CDK1, EZH2 and UMPS. In some embodiments, the cell cycle/genome integrity gene set comprises WSB2, CKS18, PAICS, RRM1, HATQ, PCNA, CDK4, GINS2, SNRPB, HIST1H4C, PTMA, SLBP, PA2G4, MAD2L2, TMX1, MLF1P, MGAT2, STMN1, CDC6, C16orf61, TOPBP1, KIAA0090, ATP5G2, YY1, PRMT5, FEN1, DAXX, UNG, MCM3, CDK2, PRKDC, LMNB1, CCNB2, GINS1, WDR76, NUSAP1, CDT1, RBL1, CDK1, EZH2 and UMPS.

In some embodiments, the stem cell/de-differentiation gene set comprises at least 1, 2, 3, 4, 5, 6 or 7 genes selected from the group consisting of SLK, CDKL5, PCDHB15, MARCH10, JPH2, PCDHB4, SOX5 and C19orf11. In some embodiments, the stem cell/de-differentiation gene set comprises SLK, CDKL5, PCDHB15, MARCH10, JPH2, PCDHB4, SOX5 and C19orf11.

In some embodiments, the method includes analyzing a cancer cell sample from the subject to determine the expression level of the genes in a gene set. As used herein, the “expression level” of the gene set the amount of a gene expression product (e.g., mRNA or protein) present in the cancer cell sample on a per cell basis.

In some embodiments, the method includes the analysis of at least two cancer cell samples acquired at different timed during EGFR TKI therapy. In some embodiments, the first sample is acquired before EGFR TKI therapy has begun. In some embodiments, the first sample is acquired within 1, 2, 3, 4, 5, 6, 7, 8, 9, 10, 11 or 12 weeks of starting EGFR TKI therapy. In some embodiments, the second sample is acquired after at least 1, 2, 3, 4, 5, 6, 7, 8, 9, 10, 11 or 12 weeks of starting EGFR TKI therapy. In some embodiments, the second sample is acquired at least 1, 2, 3, 4, 5, 6, 7, 8, 9, 10, 11, 12, 13, 14, 15, 16, 17, 18, 19, 20, 21, 22, 23 or 24 weeks after the first sample was acquired.

In some embodiments of the methods provided herein, any method can be used to determine the expression level of the genes in the gene set. For example, gene expression level can be determined by detecting the amount of a gene expression product in the sample. For example, mRNA expression product can be detected by northern blot, a nucleic acid amplification assay (e.g., RT-PCR), a real-time nucleic acid amplification assay (e.g., real-time RT-PCR), a deep sequencing assay, a dot blot or a gene expression microarray. Gene expression can also be determined by detecting the amount of protein expression product in a sample by, for example, western blot, ELISA, FACS, fluorescent microscopy or a protein expression microarray. In some embodiments, the expression of the genes in the gene set are determined using a nucleic acid or a protein microarray.

In some embodiments, the analysis of the subject sample comprises performing a nucleic acid amplification process on the sample. In certain embodiments, gene set expression product (e.g., mRNA) is amplified in the cancer cell sample using a nucleic acid amplification process. Examples of nucleic acid amplification processes include, but are not limited to, polymerase chain reaction (PCR), LATE-PCR a non-symmetric PCR method of amplification, ligase chain reaction (LCR), strand displacement amplification (SDA), transcription mediated amplification (TMA), self-sustained sequence replication (3SR), Qκ replicase based amplification, nucleic acid sequence-based amplification (NASBA), repair chain reaction (RCR), boomerang DNA amplification (BDA) and/or rolling circle amplification (RCA).

In some embodiments, the analysis of the subject sample comprises contacting the sample with a nucleic acid probe that hybridizes to a gene set, mRNA or cDNA sequence or an amplification product generated through the amplification of gene set mRNA or cDNA sequence. In certain embodiments, the nucleic acid probe comprises a nucleic acid sequence that is complementary to a nucleic acid sequence of gene set mRNA or cDNA sequence or an amplification product thereof. In some embodiments, the nucleic acid probe comprises at least 10, 15, 20, 25 or 30 nucleic acids that are complementary to a nu of nucleic acid sequence of gene set mRNA or cDNA sequence or an amplification product thereof. In some embodiments, the nucleic acid probe comprises a detectable label. In some embodiments, the detectable label is a fluorescent label, a luminescent label, an enzymatic label or a radioactive label. In some embodiments the nucleic acid probe is a molecular beacon, a molecular torch, a TaqMan probe or a scorpion probe. In some embodiments, the nucleic acid probe is immobilized on a solid support. In certain embodiments, the solid support is a bead. In some embodiments, the solid support is a part of a nucleic acid microarray.

In some embodiments, the analysis of the subject sample comprises performing a deep sequencing assay. In a deep sequencing assay, the coverage of the sequencing assay is sufficient that the same nucleic acid region is sequenced multiple times. Deep sequencing therefore allows for the quantitation of the frequency at which a particular nucleic acid sequence appears in a sample. Nucleic acid sequencing processes that can be applied to deep sequencing include, but are not limited to, chain termination sequencing, sequencing by ligation, sequencing by synthesis, pyrosequencing, ion semiconductor sequencing, single-molecule real-time sequencing, 454 sequencing, and/or Dilute-‘N’-Go sequencing.

In some embodiments, the expression level of a gene set is determined by detecting the amount of gene set expression product protein in the sample. In some embodiments, the analysis of the subject sample comprises the step of contacting the sample with an antibody or antigen binding fragment thereof. In some embodiments, the antibody or antigen binding fragment thereof is linked (either directly or indirectly) to a detectable label. In some embodiments, the detectable label is a fluorescent label, a luminescent label, an enzymatic label or a radioactive label. In some embodiments, the antibody or antigen binding fragment thereof is immobilized on a solid support. In certain embodiments, the solid support is a bead. In some embodiments, the solid support is a part of a protein microarray.

### Predicting the Development of EGFR T790M Mutation

In some aspects, provided herein is a method for predicting whether a subject with a cancer will develop an EGFR^{T790M} mutation based on the expression levels of SNORA53 (mRNA sequence available as NCBI reference number NR_{—}003015.1) and SDC2 (mRNA sequence available as NCBI reference number NM_{—}002998.3) in a cancer cell sample, as normalized to a set of housekeeping genes. In some embodiments, the method includes calculating a T790M score based on the normalized expression of SNORA53 and SDC2 in order to predict whether the subject will develop a EGFR^{T790M} mutation. In some embodiments a logistic regression model with a LASSO penalty is used to predict whether the subject will develop a EGFR^{T790M} mutation.

In some embodiments, the T790M score is calculated based on the following equation:

$$T 790 M score = \frac{{e}^{- 349.07 - 13.50 \times SNORA 53 + 50.54 \times SDC 2}}{1 + {e}^{- 349.07 - 13.50 \times SNORA 53 + 50.54 \times SDC 2}}$$

wherein SNORA53 represents the normalized expression of SNORA53 and SDC2 represents the normalized expression of SDC2. In some embodiments, a T790M score of greater than 0.5 predicts that the subject will acquire a EGFR^{T790M} mutation. In some embodiments, a T790M score of less than 0.5 predicts that the subject will not acquire a EGFR^{T790M} mutation.

In some embodiments of the methods provided herein, the set of housekeeping genes comprises 1, 2, 3, 4, 5, 6, 7, 8, 9, 10 or more genes selected from the group consisting of C15orf24 (mRNA sequence available as NCBI reference number NM_{—}020154.2), C1orf43 (mRNA sequence available as NCBI reference number NM_{—}001098616.1), CHMP2A (mRNA sequence available as NCBI reference number NM_{—}014453.3), GPI (mRNA sequence available as NCBI reference number NM_{—}000175.3), PSMB2 (mRNA sequence available as NCBI reference number NM_{—} 001199779.1), PSMB4 (mRNA sequence available as NCBI reference number NM_{—}02796.2), RAB7A (mRNA sequence available as NCBI reference number NM_{—}004637.5), REEP5 (mRNA sequence available as NCBI reference number NM_{—}005669.4), SNRPD3 (mRNA sequence available as NCBI reference number NM_{—}001278656.1), VCP (mRNA sequence available as NCBI reference number NM_{—}007126.3) and VPS29 (mRNA sequence available as NCBI reference number NM_{—}001282150.2). In some embodiments the set of housekeeping genes comprises C15orf24, C1orf43, CHMP2A, GPI, PSMB2, PSMB4, RAB7A, REEP5, SNRPD3, VCP and VPS29.

In some embodiments of the methods provided herein, any method can be used to determine gene expression. In some embodiments, gene expression is determined by a process that comprises performing a nucleic acid amplification process on the sample. In some embodiments, gene expression is determined by a process that comprises contacting the sample nucleic acid probes that hybridize to a SNORA53 mRNA sequence (e.g., a nucleic acid probe, such as a detectably labeled nucleic acid probe and/or a nucleic acid probe immobilized on a solid support) and the expression level of SDC2 is determined by a process that comprises contacting the sample nucleic acid probes that hybridize to a SDC2 mRNA sequence (e.g., a nucleic acid probe, such as a detectably labeled nucleic acid probe and/or a nucleic acid probe immobilized on a solid support). In some embodiments, gene expression is determined by a process that comprises performing a deep sequencing assay. In some embodiments, gene expression is determined using a gene expression microarray or a protein expression microarray.

In some embodiments, the method includes analyzing a cancer cell sample from the subject to determine the expression level of a gene. As used herein, the “expression level” of the gene is the amount of a gene expression product (e.g., mRNA or protein) present in the cancer cell sample on a per cell basis.

In some embodiments of the methods provided herein, any method can be used to determine the expression level of the specified genes. For example, gene expression level can be determined by detecting the amount of a gene expression product in the sample. For example, mRNA expression product can be detected by northern blot, a nucleic acid amplification assay (e.g., RT-PCR), a real-time nucleic acid amplification assay (e.g., real-time RT-PCR), a deep sequencing assay, a dot blot or a gene expression microarray. Gene expression can also be determined by detecting the amount of protein expression product in a sample by, for example, western blot, ELISA, FACS, fluorescent microscopy or a protein expression microarray. In some embodiments, the expression of the genes are determined using a nucleic acid or a protein microarray.

In some embodiments, the analysis of the subject sample comprises performing a nucleic acid amplification process on the sample. In certain embodiments, a gene expression product (e.g., mRNA) is amplified in the cancer cell sample using a nucleic acid amplification process. Examples of nucleic acid amplification processes include, but are not limited to, polymerase chain reaction (PCR), LATE-PCR a non-symmetric PCR method of amplification, ligase chain reaction (LCR), strand displacement amplification (SDA), transcription mediated amplification (TMA), self-sustained sequence replication (3SR), Qβ replicase based amplification, nucleic acid sequence-based amplification (NASBA), repair chain reaction (RCR), boomerang DNA amplification (BDA) and/or rolling circle amplification (RCA).

In some embodiments, the analysis of the subject sample comprises contacting the sample with a nucleic acid probe that hybridizes to an mRNA or cDNA expression product sequence or an amplification product generated through the amplification of a mRNA or cDNA sequence. In certain embodiments, the nucleic acid probe comprises a nucleic acid sequence that is complementary to a nucleic acid sequence of an mRNA or cDNA expression product sequence or an amplification product thereof. In some embodiments, the nucleic acid probe comprises at least 10, 15, 20, 25 or 30 nucleic acids that are complementary to a nu of nucleic acid sequence of the mRNA or cDNA sequence or an amplification product thereof. In some embodiments, the nucleic acid probe comprises a detectable label. In some embodiments, the detectable label is a fluorescent label, a luminescent label, an enzymatic label or a radioactive label. In some embodiments the nucleic acid probe is a molecular beacon, a molecular torch, a TaqMan probe or a scorpion probe. In some embodiments, the nucleic acid probe is immobilized on a solid support. In certain embodiments, the solid support is a bead. In some embodiments, the solid support is a part of a nucleic acid microarray.

In some embodiments, the analysis of the subject sample comprises performing a deep sequencing assay. In a deep sequencing assay, the coverage of the sequencing assay is sufficient that the same nucleic acid region is sequenced multiple times. Deep sequencing therefore allows for the quantitation of the frequency at which a particular nucleic acid sequence appears in a sample. Nucleic acid sequencing processes that can be applied to deep sequencing include, but are not limited to, chain termination sequencing, sequencing by ligation, sequencing by synthesis, pyrosequencing, ion semiconductor sequencing, single-molecule real-time sequencing, 454 sequencing, and/or Dilute-‘N’-Go sequencing.

In some embodiments, the expression level of a gene set is determined by detecting the amount of a gene expression product protein in the sample. In some embodiments, the analysis of the subject sample comprises the step of contacting the sample with an antibody or antigen binding fragment thereof. In some embodiments, the antibody or antigen binding fragment thereof is linked (either directly or indirectly) to a detectable label. In some embodiments, the detectable label is a fluorescent label, a luminescent label, an enzymatic label or a radioactive label. In some embodiments, the antibody or antigen binding fragment thereof is immobilized on a solid support. In certain embodiments, the solid support is a bead. In some embodiments, the solid support is a part of a protein microarray.

### EGFR Tyrosine Kinase Inhibitors

In certain embodiments, the methods provided herein relate to the treatment of cancer using an EGFR tyrosine kinase inhibitor (EGFR TKI). EGFR TKIs include any agent that inhibits the tyrosine kinase activity of EGFR. EGFR TKIs include, for example, small molecules, antibodies, antibody fragments, proteins and peptides that inhibit the tyrosine kinase activity of EGFR.

In some embodiments, the EGFR TKI is a small molecule. Examples of small molecule EGFR TKIs include gefitinib, erlotinib, afatinib, lapatinib, dacomitinib and AP26113.

Gefitinib is marketed under the trade name Iressa for the treatment of cancers associated with elevated EGFR activity, including certain breast and lung cancers. Gefitinib has the following chemical structure:

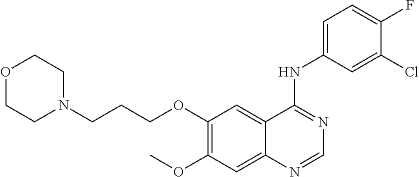 [SMILES: COc1cc2ncnc(Nc3ccc(F)c(Cl)c3)c2cc1OCCCN1CCOCC1]

Erlotinib (erlotinib hydrochloride) is marketed under the trade name Tarceva for the treatment of cancers associated with elevated EGFR activity, including NSCLC and pancreatic cancer. Erlotinib has the following chemical structure:

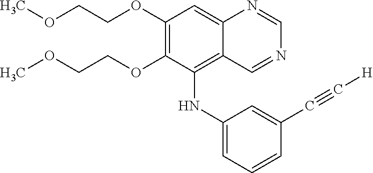 [SMILES: C#Cc1cccc(Nc2c(OCCOC)c(OCCOC)cc3ncncc23)c1]

Afatinib is marketed under the trade names Gilotrif, Tomtovok and Tovok for the treatment of cancers associated with elevated EGFR activity, including NSCLC. Afatinib has been shown to inhibit the tyrosine kinase activity of EGFR mutants carrying the T790M mutation and other mutations associated with resistance to gefitinib and erlotinib. In certain embodiments, AP267113 is administered to a subject who is predicted to have an increased likelihood of becoming resistant to EGFR TKI therapy, for example, by acquiring an EGFR^{T790M} mutation. Afatinib has the following chemical structure:

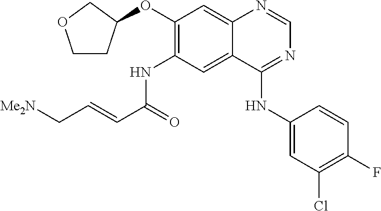 [SMILES: CN(C)C/C=C/C(=O)Nc1cc2c(Nc3ccc(F)c(Cl)c3)ncnc2cc1O[C@H]1CCOC1]

Lapatinib (lapatinib ditosylate) is marketed under the trade names Tykerb and Tyverb for the treatment of breast cancer and other solid tumors. Lapatinib inhibits the tyrosine kinase activity of both EGFR and HER2/neu. Lapatinib has the following chemical structure:

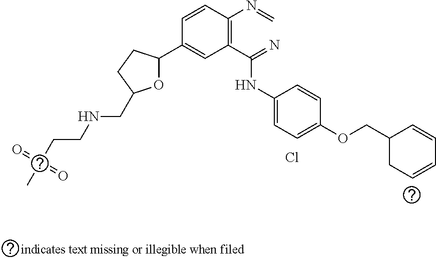

AP26113 is an EGFR TKI developed by ARIAD Pharmaceuticals that has been shown to be able to inhibit the tyrosine kinase activity of EGFR mutants carrying the T790M mutation. In certain embodiments, AP267113 is administered to a subject who is predicted to have an increased likelihood of becoming resistant to EGFR TKI therapy, for example, by acquiring an EGFR^{T790M} mutation. AP26113 has the following chemical structure:

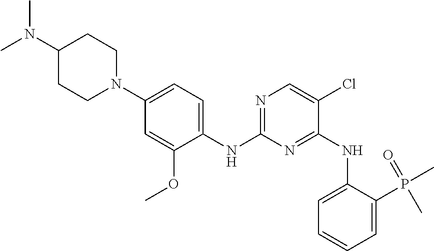

Dacomitinib is an EGFR TKI developed by Pfizer that has been shown to be able to inhibit the tyrosine kinase activity of EGFR mutants carrying the T790M mutation. In certain embodiments, dacomitinib is administered to a subject who is predicted to have an increased likelihood of becoming resistant to EGFR TKI therapy, for example, by acquiring an EGFR^{T790M} mutation. Dacomitinib has the following chemical structure:

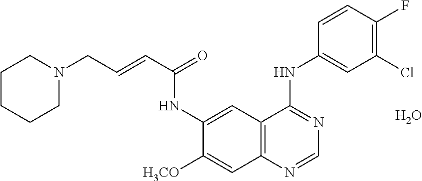

In some embodiments, the EGFR TKI is an antibody that binds to and inhibits the activity of EGFR. As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multi-specific antibodies (e.g., bispecific antibodies), single-chain antibodies and antigen-binding antibody fragments. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies. Examples of EGFR TKI antibodies include cetuximab, panitumumab, zalutumumab, nimotuzumab, necitumumab, RO5083945, ABT-806 and matuzumab.

### Therapeutic Methods

Disclosed herein are novel therapeutic methods for the treatment of cancer in a subject through the administration of an EGFR TKI. In some embodiments, the subject has a cancer that is likely to respond to the EGFR TKI. In some embodiments, the subject has a cancer that is unlikely to become resistant to the EGFR TKI. In some embodiment, the subject has been identified using a diagnostic and/or prognostic method disclosed herein as having a cancer that is likely to respond to the EGFR TKI and/or is unlikely to become resistant to the EGFR TKI. In some embodiments, an EGFR TKI that is less prone to resistance is administered to a subject who has a cancer that is likely to become resistant to other EGFR TKIs.

In some embodiments the methods provided herein can be used to treat any cancer. In some embodiments, the cancer carries an EGFR activating mutation (e.g., the cancer carries a mutation that results in the overexpression or constitutive activation of EGFR). In some embodiments, the cancer is a non-small cell lung cancer (NSCLC), an anal cancer or glioblastoma multiforme. In some embodiments, the cancer is a cancer from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer carries a EGFR^{T790M} mutation.

In some embodiments, the agents provided herein can be administered in combination therapy, i.e., combined with other agents. For example, in some embodiments, when used for treating cancer, the methods provided herein comprise administering a composition described herein in conjunction with one or more chemotherapeutic agents. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

Conjunctive therapy includes sequential, simultaneous and separate, and/or co-administration of the active compounds in a such a way that the therapeutic effects of the first agent administered have not entirely disappeared when the subsequent agent is administered. In certain embodiments, the second agent may be co-formulated with the first agent or be formulated in a separate pharmaceutical composition.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds provided herein employed in the pharmaceutical composition at levels lower than that required in order to achieve the desired therapeutic effect and gradually increase the dosage until the desired effect is achieved.

### Examples

The invention now being generally described will be more readily understood by reference to the following examples which are included merely for purposes of illustration of certain aspects and embodiments of the present invention, and are not intended to limit the invention in any way.

### Experimental Methods

### General

Paired tumor biopsy samples from 16 patients with EGFR-mutant lung adenocarcinoma before anti-EGFR TKI therapy and following the acquisition of resistance were analyzed. Biopsies were obtained from patients who provided written informed consent. Whole exome sequencing was performed on DNA extracted from all 16 tumor biopsy pairs, as well as on normal from matched, non-tumorous tissue, as described previously in Gnirke et al., Nature Biotechnology 27:182-189 (2009), with paired-end reads of 100 bp on the Illumina HiSeq 2500 platform. In addition, targeted cancer gene sequencing was carried out using the Illumina TruSeq All-Cancer Panel (TSACP) in order to validate findings in the exome data and to supplement regions of the exome poorly covered due to FFPE-related artifact. RNA was available for 13 of the 16 tumor pairs, and whole transcriptome sequencing was performed to generate paired-end reads of 100 bp on the Illumina HiSeq 2500 platform.

### Tissue Collection and DNA/RNA Isolation

Following histopathologic review, formalin-fixed, paraffin embedded tissue (FFPE) tissue blocks were selected for DNA and RNA extraction, which was performed at SeqWright, Inc (Houston, Tex.). DNA and RNA were extracted from FFPE tissues using the Qiagen Allprep DNA/RNA FFPE kit, and non-tumorous peripheral blood underwent DNA extraction using the QIAmp DNA Mini kit according to the manufacturer's instructions. DNA and RNA quantities were determined by fluorometry using the Qubit 2.0 system and by NanoDrop spectrophotometry. DNA quality was assessed by agarose gel electrophoresis and RNA quality was assessed by Agilent 2100 Bioanalyzer. DNA samples requiring whole genome amplification (WGA) due to low quantity (less than 500 ng) were processed with the Qiagen REPLI-g Mini kit.

### Whole Exome and Targeted Cancer Panel Sequencing

Whole exome sequencing (WES) and targeted gene sequencing were performed at SeqWright, Inc (Houston, Tex.). Isolated genomic DNA underwent exome enrichment using the Agilent SureSelect Human All Exon v5 kit, according to the manufacturer's instructions, and libraries for each sample were run on the Illumina HiSeq 2500 platform to generate 100 bp paired-end reads, with a mean coverage of 59×. Targeted cancer gene sequencing was performed using the Illumina TruSeq Amplicon—Cancer Panel (TSACP) kit, which consists of a highly multiplexed PCR reaction that generates 212 amplicons spanning 48 cancer genes of interest. 250 ng of input tumor DNA was processed for each sample according to the manufacturer's instructions. Amplicons were run on the Illumina MiSeq system to achieve a mean sequencing depth of 6900×.

### Whole Transcriptome/RNA Sequencing (RNA-Seq)

Whole transcriptome sequencing was performed at SeqWright, Inc (Houston, Tex.). Total RNA isolated from FFPE tumor tissue first underwent ribosomal RNA depletion using the RiboMinus kit (Life Technologies) according to the manufacturer's instructions. The rRNA-depleted RNA for each sample was then processed using Illumina's TruSeq Stranded mRNA Sample Prep Kit according to the manufacturer's instructions in order to generate strand-specific RNA-Seq libraries. The libraries were run on the Illumina HiSeq 2500 platform to generate a mean of 69 million reads across all the patient samples.

### Example 1

### Molecular Alterations in Genes that Promote Acquired EGFR TKI Resistance or Tumor Initiation or Progression

Sixteen clinical cases of advanced stage EGFR-mutant NSCLC were selected for molecular analysis based on: 1) an established clinical response and resistance to an approved EGFR TKI (erlotinib or gefitinib); 2) adequate formalin-fixed paraffin-embedded (FFPE) tissue from both the pre-treatment and post-resistance specimen; 3) matched normal material (peripheral blood or non-tumor FFPE tissue); 4) appropriate informed consent and local institutional review board (IRB) approval. Each patient in our cohort demonstrated a radiographic and clinical response to initial EGFR TKI therapy and met established criteria for acquired EGFR TKI resistance. The clinical and pathological characteristics of these patients accurately represent established characteristics of EGFR-mutant NSCLC, enriched for lung adenocarcinoma, female gender, and no tobacco exposure. In this cohort, 11 patients (69%) were female and 5 were male (31%), with an average of 56.2 years and an average time to progression (TTP) of 15.4 months, consistent with prior studies. The longest TTP observed was 40 months, while the shortest was 3.8 months. Five patients (31%) were active or former smokers at presentation, 8 (50%) were never-smokers, with the smoking status of the remaining 3 cases unknown. All paired biopsies were reviewed microscopically to assess for histologic transformation to small cell carcinoma, previously reported in some EGFR TKI resistant NSCLCs. No morphologic transformations were observed in these resistant cases.

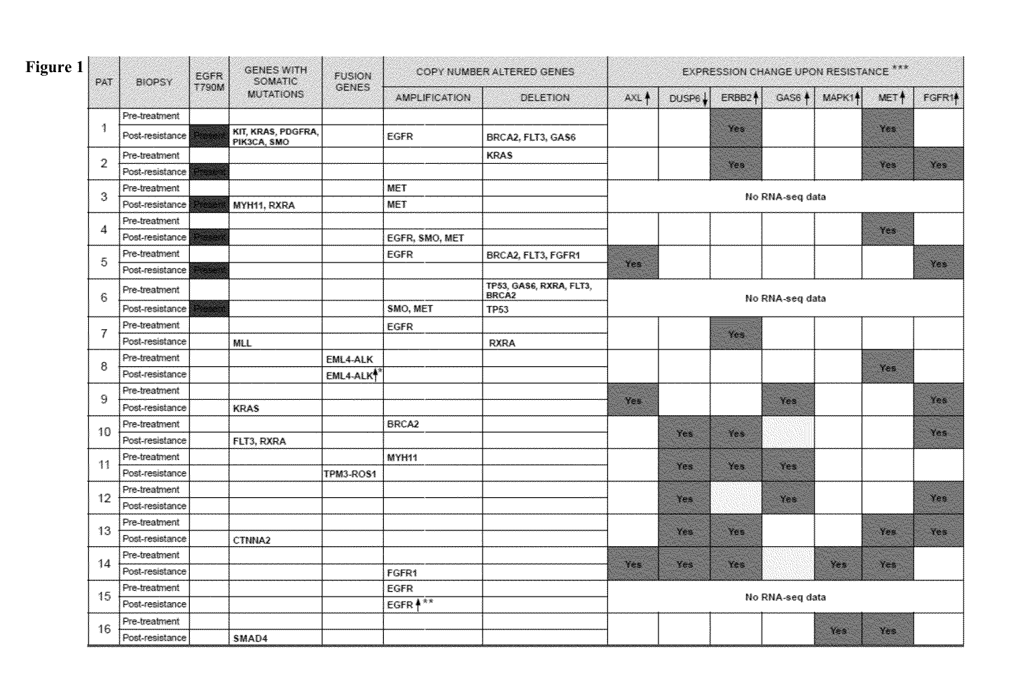

Using the whole exome sequencing (WES) and targeted exome (TE) deep sequencing data, the somatic mutation and copy number alteration status of genes previously established as drivers of acquired EGFR TKI resistance in EGFR-mutant NSCLC or with known functions in the pathogenesis of human cancers more broadly were interrogated (FIG. 1). The EGFR^{T790M} resistance mutation was identified in 6 resistant cases and none of the pre-treatment specimens (FIG. 1). Somatic mutations that have not previously been described in EGFR TKI resistant NSCLC and that represent potentially actionable therapeutic targets were also identified. These novel mutations include activating mutations in KRAS (Q61K and A146T), PIK3CA (R108H), KIT (D579Y), and SMO (W535R), and an uncharacterized mutation within the kinase domain of PDGFRA (T894S) (FIG. 1). Notably, several of these mutations co-occurred with EGFR^{T790M} in a single resistant specimen with many harboring multiple somatic alterations, highlighting the complex heterogeneity of the somatic mutation landscape in this cohort of EGFR TKI resistant clinical cases.

The exome sequencing data was used to investigate somatic copy number alterations (SCNAs) in these specimens. Focal genomic amplification of MET was present in 2 of the resistance specimens, with a corresponding increase in MET mRNA expression in one of these two cases. Both of these MET-amplified resistant specimens also acquired an EGFR^{T790M} mutation. Novel genomic amplifications not previously associated with EGFR TKI resistance in EGFR-mutant NSCLC and that could contribute to resistance were identified, including amplification of the receptor kinase FGFR1 and of SMO which are each potentially clinically actionable events.

Next the whole transcriptome sequencing data was used to identify fusion genes present in these specimens that could be targeted with an approved targeted therapy. In one resistant specimen but not the corresponding pre-treatment tumor, evidence of a chimeric transcript was found that included the kinase domain of ROS1 and TPM3, a fusion observed previously in NSCLC, but never before in a tumor with acquired EGFR TKI resistance. Moreover, an additional case (Patient #8) was found to have a canonical EML4-ALK fusion transcript in both pre-treatment and therapy resistant tumor, with increased EML4-ALK mRNA expression in the resistant specimen. Although activating EGFR mutations and EML4-ALK fusions can occur at very low frequency prior to therapy, these findings indicate that these NSCLC driver kinases can co-exist and may evolve as distinct sub-clones during EGFR TKI therapy resistance.

Next, the pre-treatment and resistant tumor transcriptomes were examined in each patient for evidence of gene expression changes that occur in the setting of acquired EGFR TKI resistance in EGFR-mutant NSCLC. Specifically, whether the resistant tumors harbored increased levels of MET, ERBB2, GAS6, AXL, MAPK1, and FGFR1, and decreased levels of DUSP6 in comparison to the corresponding pre-treatment specimen was examined Increased AXL, or GAS6, or both, was found in 5 resistant cases, consistent with prior studies. While only 2 cases exhibited genomic amplification of MET at resistance, 7 demonstrated increased MET mRNA expression. While no cases exhibited a statistically significant genomic amplification of ERBB2 at resistance, 7 cases were found to have increased ERBB2 mRNA expression. Evidence of MAPK pathway activation in several resistant specimens through either downregulation of DUSP6 or upregulation of MAPK1 was observed. Since we observed the presence of FGFR1 genomic amplification in one case, examined the levels of FGFR1 mRNA expression were also examined in the post-resistance biopsies compared to the corresponding pre-treatment tumor specimen. Increased FGFR1 expression was observed in 6 cases. Together, the exome and transcriptome data reveal previously unappreciated molecular complexity present in EGFR TKI clinical resistance in EGFR-mutant NSCLC.

### Example 2

### Molecular Classification of EGFR TKI Resistance Through Whole Transcriptome Expression Analysis

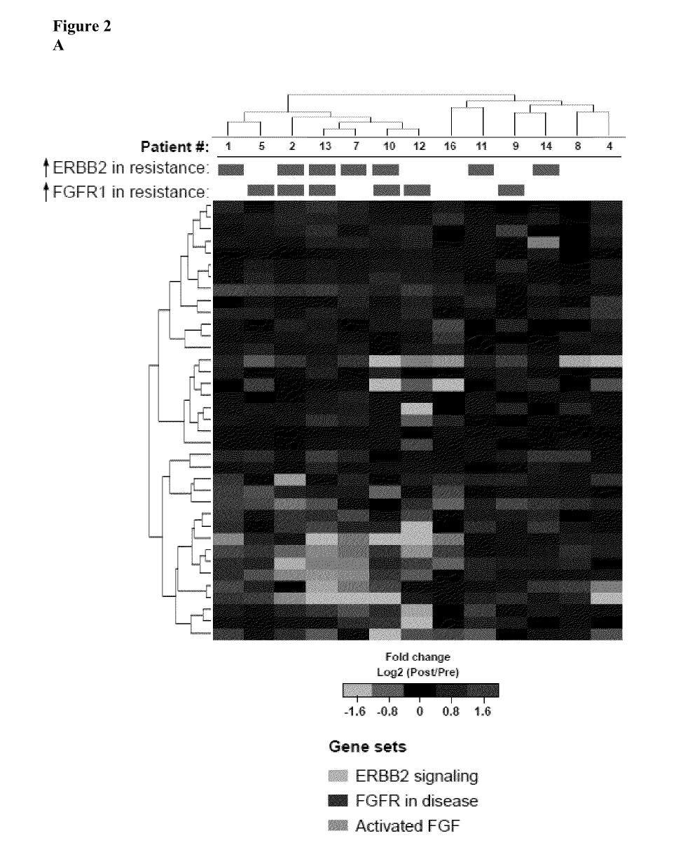

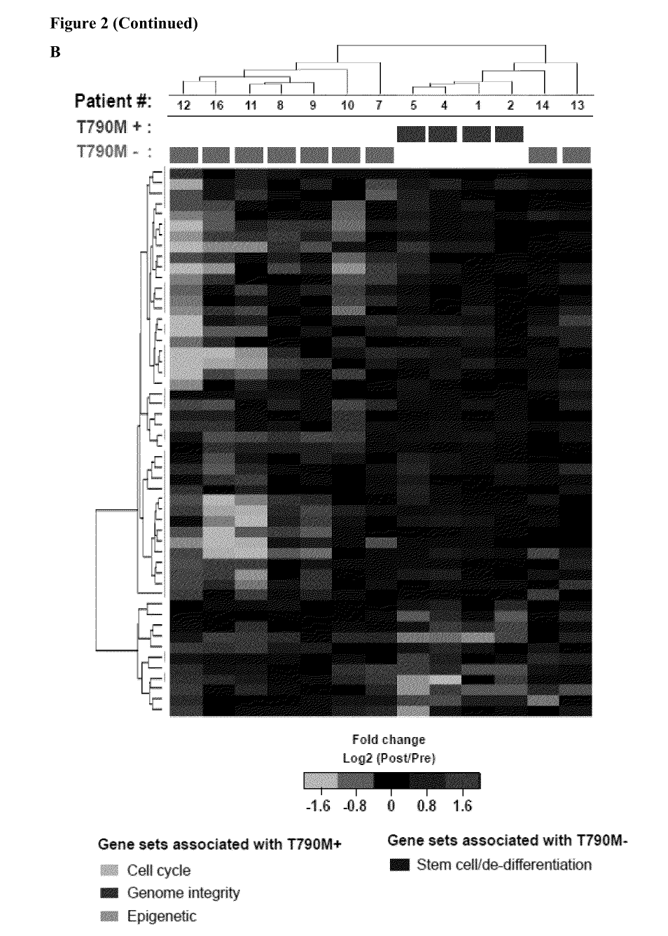

Whether whole transcriptome analysis could provide insight into the underlying biological basis of therapeutic resistance and of the selective emergence of a particular mechanism(s) of resistance was examined First, which sets of genes were differentially expressed in the therapy-resistant versus pre-treatment tumor specimens were tested. As a group, the EGFR TKI-resistant biopsies demonstrated increased ERBB2 and FGFR pathway signaling when compared to the pre-treatment samples (FIG. 2A). Specific genes belonging to these gene sets that demonstrated an increase in expression post resistance included the PI3-K pathway genes AKT1, AKT2, and the MAPK pathway gene HRAS, while the mRNA expression of the tumor suppressor PTEN that negatively regulates PI3-K signaling was decreased. These data indicate that although the emergence of EGFR TKI resistance can occur through many discrete and often overlapping genetic alterations, these molecular events could drive resistance through the activation of a smaller number of core biological pathways that could be inhibited with available clinical targeted therapies.

Next, whether a specific biological program(s) could be associated with the selective, recurrent emergence of a specific resistance-driving alteration during EGFR targeted therapy was investigated. Since the EGFR^{T790M} mutation is the most common resistance-conferring genetic alteration, which gene sets were differentially expressed in the EGFR^{T790M} positive biopsies were examined when compared to the corresponding pre-treatment tumor specimens. This analysis revealed upregulation of genes underlying cell cycle progression and genome integrity maintenance (FIG. 2B), cellular processes that are often linked, suggesting a requirement for these biological programs during therapy resistance that is associated specifically with the presence of the EGFR^{T790M} mutation. In contrast, this gene expression profiling analysis revealed that the EGFR^{T790M} negative cases exhibited upregulation of gene sets associated with stem cell and de-differentiation biological programs (FIG. 2B). Together, these data indicate that distinct biological processes could underlie the evolution of distinct classes of EGFR TKI resistance (EGFR^{T790M} positive versus negative) in EGFR-mutant NSCLC patients.

### Example 3

### Molecular Characterization of Genome Maintenance Programs in EGFR TKI Resistance

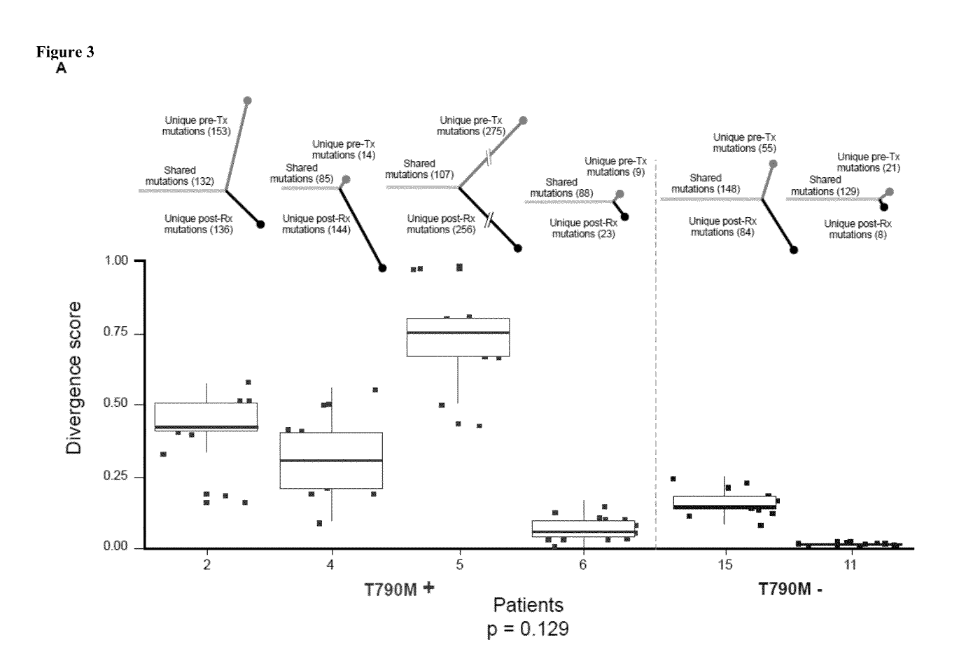

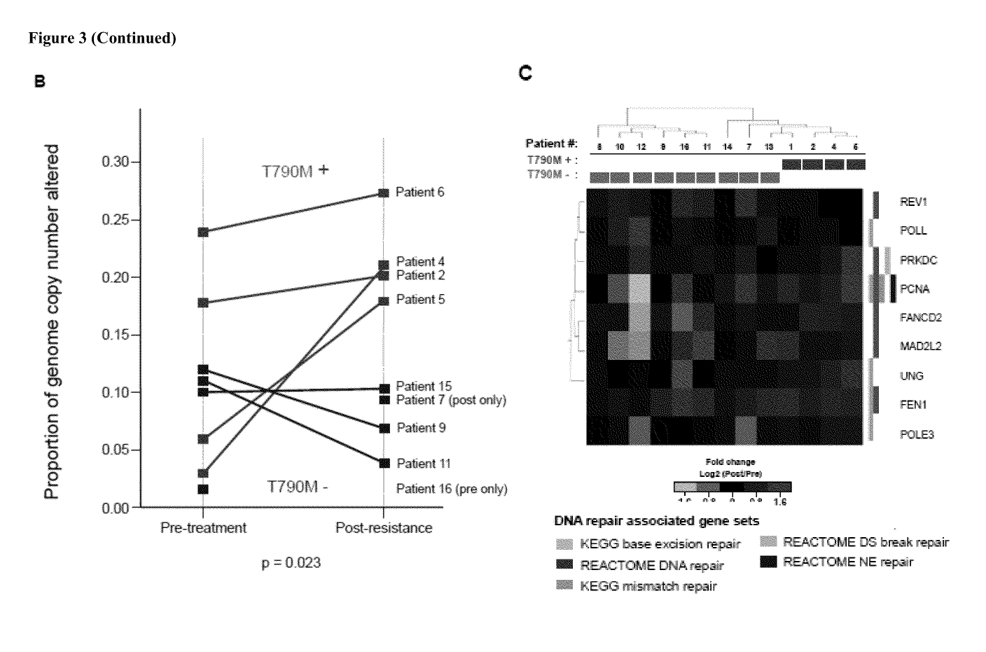

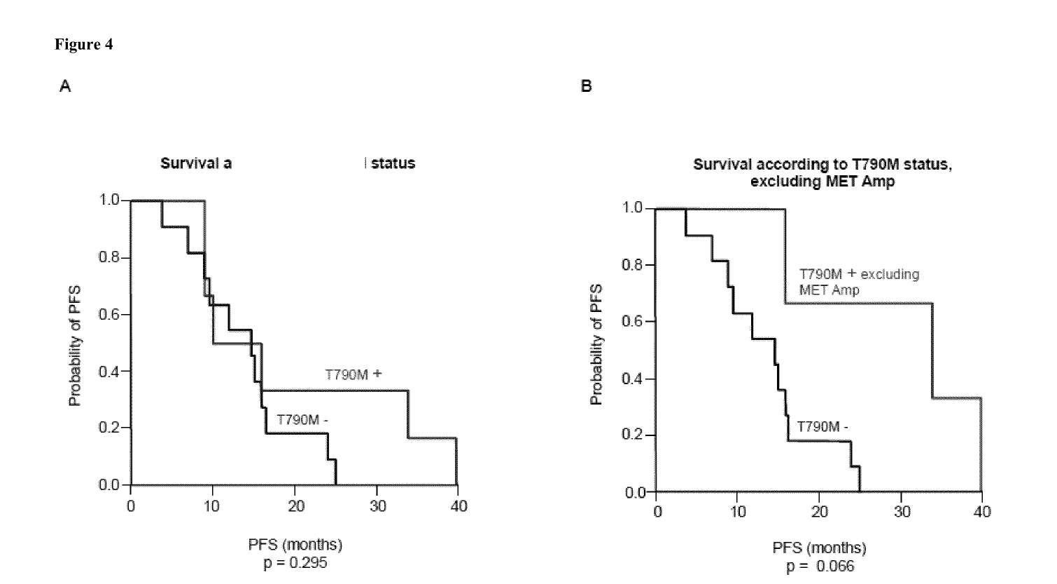

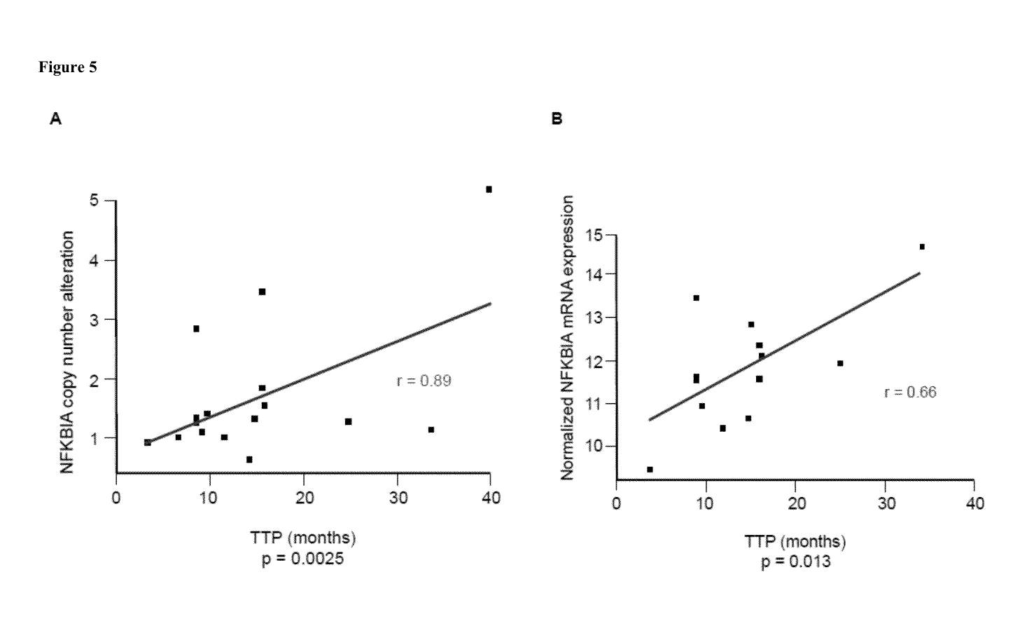

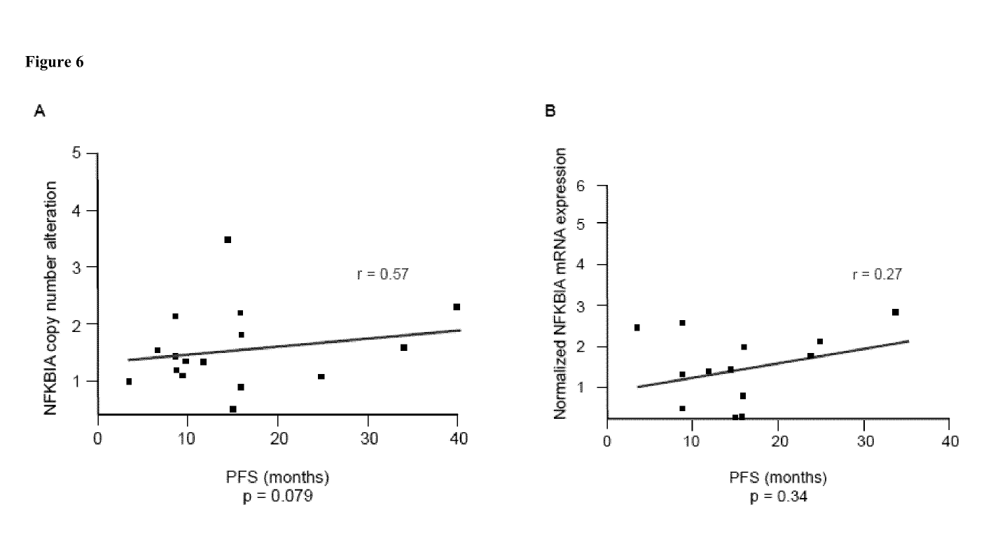

Given the observed association of increased cell cycle progression and genome integrity maintenance programs with the presence of the EGFR^{T790M} mutation in the resistant tumor specimens, it was reasoned that these cases, but not those without the EGFR^{T790M} mutation, would exhibit evidence of increased genetic divergence and genomic instability when compared to the corresponding pre-treatment tumor specimens. To test this hypothesis, the degree to which each resistant tumor diverged from the corresponding treatment-naïve specimen was examined by measuring somatic mutational divergence using the WES data. In aggregate, the tumors with EGFR^{T790M} mutations exhibited increased clonal divergence as indicated by a higher ratio of the presence of unique to shared mutations in the paired biopsies (FIG. 3A). Interestingly, this trend did not follow a consistent pattern of clonal divergence, as some cases such as patients #4 and #6 had very few mutations unique to their pre-treatment tumor biopsies, while others including patients #2 and #5 had greater unique variants in their pre-treatment tumor specimens. Furthermore, this trend towards an association between increased clonal divergence and the presence of the EGFR^{T790M} mutation reflected an altered genetic background but not an increased number of somatic mutations, as the overall somatic mutation rate was consistent across this tumor cohort.

Next, it was examined whether, in addition this increased clonal divergence, the cases with EGFR^{T790M} were also associated with increased genomic instability, as measured by the degree of copy number alterations present in each resistant versus pre-treatment tumor genome. Indeed, EGFR^{T790M} positive resistant tumors demonstrated a significantly higher level of copy number alterations when compared to the EGFR^{T790M} negative resistant tumors (FIG. 3B). Furthermore, the EGFR^{T790M} positive, but not negative, cases exhibited a trend towards increased copy number alterations during the development of therapeutic resistance (FIG. 3B).

Based on these data indicating an altered genetic background (increased clonal divergence) and a striking increase in copy number alterations in the genomes of the EGFR^{T790M} positive versus negative cases, it was hypothesized that EGFR^{T790M} positive cases would exhibit increased expression of genes and gene sets that promote DNA repair in comparison with the EGFR^{T790M} negative cases. By examining the expression of several DNA repair associated gene sets, it was determined that EGFR^{T790M} positive cases exhibited increased expression of genes involved in multiple DNA repair pathways, including PCNA, which plays a critical role in DNA damage tolerance (DDT), and PRKDC, which functions in the repair of double-stranded DNA breaks (P<0.05, FIG. 3C). Collectively, these data suggest that the evolution of EGFR^{T790M}-mediated resistance is associated with biological programs that facilitate cell cycle progression and concurrent genome maintenance during substantial genetic divergence and instability that occurs in the tumor genome during EGFR TKI therapy.

### Example 4

### Impact of the Genetic Landscape of EGFR TKI Resistance on Clinical Outcome

Having characterized the somatic molecular events that could contribute to clinical resistance, it was next investigated which molecular abnormalities in either the pre-treatment or post-resistance biopsies correlated clinical outcomes in these patients, as measured by time to progression (TTP) on EGFR TKI therapy. Patients with the EGFR^{T790M} mutation exhibited a trend towards longer TTP, a finding that was accentuated when the two patients harboring a MET genomic amplification concurrent with EGFR^{T790M} were removed from the analysis (FIG. 4). It was examined which recurrent genetic events present in the datasets were significantly correlated with outcome. No individual somatic mutation was associated significantly with TTP in these patients, likely given that none were highly recurrent in this cohort. While several copy number alterations were correlated significantly with TTP, only a subset of the genes within these copy number alterations also exhibited the corresponding change in mRNA expression. Among these genes, coordinated amplification and increased mRNA expression of NFKBIA, an inhibitor of NF-κB signaling, was most strongly associated with clinical outcome, particularly in the resistant tumor specimens (FIG. 5A-B, FIG. 6). These findings suggest that decreased NF-κB signaling in EGFR-mutant NSCLC cells is associated with improved response to EGFR TKI therapy. These findings nominate NFKBIA genomic amplification, together with increased NFKBIA expression, as a biomarker and therapeutic target to enhance EGFR TKI clinical responses.

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a subject who has a cancer in which NFKBIA is overexpressed or has an elevated copy number comprising administering to the subject an effective amount of gefitinib, erlotinib or afatinib.

2. The method of claim 1, further comprising the step of analyzing a cancer cell sample from the subject to determine the expression level or gene copy number of NFKBIA before administering to the subject an effective amount of gefitinib, erlotinib or afatinib.

3. The method of claim 1, wherein the cancer carries a mutation that results in the overexpression or constitutive activation of EGFR.

4. The method of claim 3, wherein the cancer is non-small cell lung cancer.

5. The method of claim 1, wherein the overexpression or elevated copy number of NFKBIA is relative to NFKBIA expression in non-cancer cells of the same tissue type as the cancer cells.

6. The method of claim 2, wherein the analysis of the cancer cell sample comprises performing a nucleic acid amplification process on the sample, contacting the sample with a nucleic acid probe that hybridizes to a NFKBIA genomic DNA or mRNA sequence, performing a deep sequencing assay or contacting the sample with an anti-NFKBIA antibody or antigen binding fragment thereof.

7. A method for determining whether a cancer carrying an EGFR activating mutation in a subject is resistant to an EGFR tyrosine kinase inhibitor (EGFR TKI), the method comprising the steps of:
a) determining the expression level of a cell cycle/genome integrity gene set in a first cancer cell sample obtained before the subject began EGF TKI therapy;
b) determining the expression of the cell cycle/genome integrity gene set in a second cancer cell sample obtained after the subject had undergone EGF TKI therapy;
wherein increased expression of the cell cycle/genome integrity gene set in the second cancer cell sample compared to the first cancer cell sample indicates that the cancer in the subject is resistant to the EGFR TKI.

8. The method of claim 7, wherein the cell cycle/genome integrity gene set comprise one or more genes selected from the group consisting of: WSB2, CKS18, PAICS, RRM1, HATQ, PCNA, CDK4, GINS2, SNRPB, HIST1H4C, PTMA, SLBP, PA2G4, MAD2L2, TMX1, MLF1P, MGAT2, STMN1, CDC6, C16orf61, TOPBP1, KIAA0090, ATP5G2, YY1, PRMT5, FEN1, DAXX, UNG, MCM3, CDK2, PRKDC, LMNB1, CCNB2, GINS1, WDR76, NUSAP1, CDT1, RBL1, CDK1, EZH2 and UMPS.

9. The method of claim 7, wherein the cell cycle/genome integrity gene set comprises WSB2, CKS18, PAICS, RRM1, HATQ, PCNA, CDK4, GINS2, SNRPB, HIST1H4C, PTMA, SLBP, PA2G4, MAD2L2, TMX1, MLF1P, MGAT2, STMN1, CDC6, C16orf61, TOPBP1, KIAA0090, ATP5G2, YY1, PRMT5, FEN1, DAXX, UNG, MCM3, CDK2, PRKDC, LMNB1, CCNB2, GINS1, WDR76, NUSAP1, CDT1, RBL1, CDK1, EZH2 and UMPS.

10. The method of claim 7, wherein the cancer is non-small cell lung cancer.

11. The method of claim 7, further comprising:
i. determining the expression of a stem cell/de-differentiation gene set in the first cancer cell sample;
ii. determining the expression of the stem cell/de-differentiation gene set in the second cancer cell sample;
wherein increased expression of the cell cycle/genome integrity gene set in the second cancer cell sample compared to the first cancer cell sample and decreased expression of the stem cell/de-differentiation gene set in the second cancer cell sample compared to the first cancer cell sample indicates that the cancer in the subject is resistant to the EGFR TKI

12. The method of claim 11, wherein the stem cell/de-differentiation gene set comprises SLK, CDKL5, PCDHB15, MARCH10, JPH2, PCDHB4, SOX5 and C19orf11.

13. The method of claim 7, wherein the expression of the cell cycle/genome integrity gene set is determined using a gene expression or protein expression microarray or a deep sequencing assay.

14. The method of claim 7, wherein the EGFR TKI is gefitinib, erlotinib or afatinib.

15. A method of treating a cancer carrying an EGFR activating mutation in a subject, the method comprising the steps of:
a) obtaining a first cancer cell sample from the subject;
b) administering an EGFR tyrosine kinase inhibitor (EGFR TKI) to the subject;
c) obtaining a second cancer cell sample from the subject;
d) determining the expression of a cell cycle/genome integrity gene set in the first cancer cell sample and the second cancer cell sample; and
e) continuing administering the EGFR TKI to the subject if the cell cycle/genome integrity gene set is not expressed at a higher level in the second cancer cell sample compared to the first cancer cell sample.

16. A method for predicting whether a cancer carrying an EGFR activating mutation in a subject will develop an EGFR^{T790M} mutation, the method comprising:
a) determining the expression levels of SNORA53 and SDC2 in a cancer cell sample;
b) determining the expression level of a set of housekeeping genes in the cancer cell sample;
c) normalizing the expression level of SNORA53 and SDC2 against the mean expression of the set of housekeeping genes;
d) calculating a T790M score based on the normalized expression of SNORA53 and SDC2 to predict whether the subject will develop a EGFR^{T790M} mutation.

17. The method of claim 16, wherein the T790M score is calculated based on the following equation:
$$T 790 M score = \frac{{e}^{- 349.07 - 13.50 \times SNORA 53 + 50.54 \times SDC 2}}{1 + {e}^{- 349.07 - 13.50 \times SNORA 53 + 50.54 \times SDC 2}}$$
wherein a T790M score of greater than 0.5 predicts that the subject will acquire a EGFR^{T790M} mutation.

18. The method of claim 16, wherein the set of housekeeping genes comprises one or more genes selected from the group consisting of C15orf24, C1orf43, CHMP2A, GPI, PSMB2, PSMB4, RAB7A, REEP5, SNRPD3, VCP and VPS29.

19. The method of claim 16, wherein the cancer is non-small cell lung cancer.

20. The method of claim 16, further comprising administering to the subject a EGFR tyrosine kinase inhibitor (EGFR TKI) if the subject is not predicted to develop a EGFR^{T790M} mutation.
